(19) United States
(12) Patent Application Publication
(10) Pub. No.: US 2022/0076838 A1
(43) Pub. Date: Mar. 10, 2022

(54) ACUTE KIDNEY INJURY MONITORING

(71) Applicant: Covidien LP, Mansfield, MA (US)

(72) Inventors: Jacob D. Dove, Lafayette, CO (US); David H. Milkes, Ward, CO (US); David J. Miller, Austin, TX (US)

(21) Appl. No.: 17/308,511

(22) Filed: May 5, 2021

Related U.S. Application Data
(60) Provisional application No. 63/074,781, filed on Sep. 4, 2020.

(51) Int. Cl. G16H 50/30; G01N 33/493; G16H 50/70; G16H 10/60; G16H 40/67

(52) U.S. Cl. CPC G16H 50/30; G01N 33/493; A61B 10/007; G16H 10/60; G16H 40/67; G16H 50/70

(57) ABSTRACT

An example device includes memory and processing circuitry communicatively coupled to the memory. The processing circuitry is configured to determine a first baseline value of dissolved oxygen in a fluid and determine a second baseline value of a total oxygen output in the fluid. The processing circuitry is also configured to receive, from a first sensor, a first signal indicative of an amount of dissolved oxygen in the fluid and receive, from a second sensor, a second signal indicative of the output of the fluid. The processing circuitry is configured to determine a risk of developing acute kidney injury (AKI) based at least in part on the first baseline value, the second baseline value, the first signal, and the second signal.

This application claims the benefit of U.S. Provisional Application No. 63/074,781, entitled, “ACUTE KIDNEY INJURY MONITORING” and filed Sep. 4, 2020, the entire contents of which are hereby incorporated by reference.

### TECHNICAL FIELD

This disclosure relates to patient monitoring.

### BACKGROUND

Medical devices, such as catheters, may be used to assist a patient in voiding their bladder. In some instances, such catheters may be used during and/or after surgery. In the case of using a catheter to assist a patient in voiding their bladder, a Foley catheter is a type of catheter that may be used for longer time periods than a non-Foley catheter. Some Foley catheters are constructed of silicon rubber and include an anchoring member, which may be an inflatable balloon, that may be inflated in a patient's bladder to serve as an anchor so a proximal end of the catheter does not slip out of the patient's bladder.

### SUMMARY

In general, the disclosure describes devices, systems, and techniques for renal monitoring (also referred to herein as kidney function monitoring) based on parameters of interest associated with a fluid (e.g., urine) sensed by one or more sensors. The parameters of interest can be, for example, a substance of interest (e.g., oxygen) or a property of interest (e.g., a volume or temperature) of the fluid. In some examples, the one or more sensors are configured to sense the parameters of interest associated with a fluid in a Foley catheter, such as urine in a drainage lumen of the Foley catheter, or in a volume of the fluid separate from, but fluidically connected to the Foley catheter. In some examples, one or more of the sensors may be separate from the Foley catheter. In other examples, the sensors may be part of the Foley catheter.

In some examples, this disclosure describes devices, systems, and techniques for determining a risk that a patient may develop acute kidney injury (AKI) based at least in part on the sensed parameters which may facilitate earlier intervention by a clinician to reduce the chance that the patient may develop AKI or reduce the severity of the AKI. The devices, systems, and techniques may determine baseline(s) for parameter(s) and may compare the parameters to thresholds, which in at least some cases, may be based on the baselines.

In one example, this disclosure describes a method comprising: determining, by processing circuitry, a first baseline value of dissolved oxygen in a fluid; determining, by the processing circuitry, a second baseline value of a total oxygen output in the fluid; receiving, from a first sensor, a first signal indicative of an amount of dissolved oxygen in the fluid; receiving, from a second sensor, a second signal indicative of the output of the fluid; and determining, by the processing circuitry, a risk of developing acute kidney injury (AKI) based at least in part on the first baseline value, the second baseline value, the first signal, and the second signal.

In another example, this disclosure describes a device comprising memory; and processing circuitry communicatively coupled to the memory, the processing circuitry being configured to: determine a first baseline value of dissolved oxygen in a fluid; determine a second baseline value of a total oxygen output in the fluid; receive, from a first sensor, a first signal indicative of an amount of dissolved oxygen in the fluid; receive, from a second sensor, a second signal indicative of the output of the fluid; and determine a risk of developing acute kidney injury (AKI) based at least in part on the first baseline value, the second baseline value, the first signal, and the second signal.

In another example, this disclosure describes a device comprising memory; and processing circuitry communicatively coupled to the memory, the processing circuitry being configured to: determine at least two measures of an amount of dissolved oxygen in a fluid based on a first signal; apply, to determine a first baseline value of dissolved oxygen in the fluid, at least one of an exponential decay or a non-linear regression to the at least two measures of the amount of dissolved oxygen in the fluid; determine at least two measures of the output of the fluid based on a second signal; apply, to determine a second baseline value of a total oxygen output in the fluid, at least one of an exponential decay or a non-linear regression to at least one of: a) the at least two measures of the amount of dissolved oxygen in the fluid; b) the at least two measures of the output of the fluid based on the second signal; or c) at least two measures of the total oxygen output in the fluid based on the at least two measures of the dissolved oxygen in the fluid and the at least two measures of the amount of dissolved oxygen in the fluid; and determine a risk of developing acute kidney injury (AKI) based at least in part on the first baseline value, the second baseline value, the first signal, and the second signal.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a diagram illustrating an example medical device including first and second sensors.

FIG. 2 is a diagram illustrating an example cross-section of the medical device of FIG. 1, the cross-section being take along lines 2-2 of FIG. 1.

FIG. 3 is a block diagram of an example external device that may be used with a medical device according to the techniques of this disclosure.

FIG. 4 is a flowchart illustrating example AKI risk determination techniques according to this disclosure.

FIG. 5 is a flow diagram illustrating example techniques that include determining a risk score indicative of a risk of a patient developing acute kidney injury (AKI) based on an amount of dissolved oxygen in the urine (uPO₂) and/or urine output.

FIG. 6 is a graph illustrating example measurements of uPO2 over time, modeled initial decrease in uPO₂, and baseline uPO₂.

FIG. 7 is another graph illustrating example measurements of uPO2 over time, modeled initial decrease in uPO₂, and baseline uPO₂.

### DETAILED DESCRIPTION

Acute kidney injury (AKI) is a complication that may occur after certain medical procedures, such as some cardiac surgeries, e.g., coronary artery bypass grafting (CABG). AKI also may occur after other surgeries that are lengthy and involve significant blood loss or fluid shifts. For example, a surgery patient's body may alter where their blood is directed to, which may lead to hypoxia of a kidney. A cause of surgery-associated AKI is hypoxia of the kidneys, which may cause an ischemia reperfusion injury to a kidney of the patient. This ischemia reperfusion injury may cause degradation of renal function of the patient. The degradation of renal function may cause an accumulation of waste products in the bloodstream, which may delay the patient's recovery from the surgery and lead to more extended hospital stays and may even lead to further complications.

The present disclosure describes example devices that are configured to monitor kidney function of patients, such as patients who are undergoing or who have undergone such surgeries, which may help reduce occurrences of AKI by providing clinicians with an assessment of the risk that a specific patient may develop AKI. This may facilitate a clinician intervening prior to the patient developing AKI. For example, a clinician may initiate or make changes to hemodynamic management (e.g., blood pressure management, fluid management, blood transfusions, etc.), make changes to cardiopulmonary bypass machine settings, or avoid providing nephrotoxic drugs. Post operatively, a clinician may intervene with a Kidney Disease: Improving Global Outcomes (KDIGO) bundle or an AKI care bundle. The devices may be communicatively coupled to a plurality of sensors (e.g., two or more sensors) configured to sense different parameters of a fluid of interest, such as urine in the case of kidney function monitoring. While urine, bladders, and AKI are primarily referred to herein to describe the example devices, in other examples, the devices may be used with other target locations in a patient, such as intravascular locations, and to monitor fluids of interest other than urine and/or other patient conditions other than kidney function.

Systemic vital signs like cardiac output, blood pressure, and hematocrit are useful but may not be fully sufficient to monitor the kidneys. When the body is stressed, such as during cardiac surgery, blood flow is reduced to vital organs in a reliable sequence based on the criticality of the organs. For example, it has been observed that typically the first organ to get reduced blood flow is the skin followed by the gut, then the kidneys, then the brain, then the heart. The skin and the gut can withstand short hypoxic episodes and recover normal function, but kidney function may be adversely impacted by even brief hypoxic episodes.

Example sensed parameters that may be useful in determining the state of kidney function include, but are not limited to, any one or more of urine output (e.g., flow rate or volume), amount of dissolved oxygen in the urine (oxygen tension or uPO₂), total oxygen in the urine, the trend of the amount of dissolved oxygen in the urine (oxygen tension or uPO₂), and the trend of the total oxygen in the urine. Other sensed parameters that may be useful in determining the state of kidney function may include urine or bladder temperature, urine concentration (urine osmolarity), amount of dissolved carbon dioxide in the urine, urine pH, bladder or abdominal pressure, urine color, urine creatinine, urine electrical conductivity, urine sodium, or motion from an accelerometer or other motion sensor.

For example, the amount of dissolved oxygen in a patient's urine may be indicative of kidney function or kidney health. Dissolved oxygen in a patient's urine and bladder may correlate to perfusion and/or oxygenation of the kidneys, which is indicative of kidney performance.

One approach to monitoring a patient for an increased risk of AKI is to monitor the oxygenation status of the kidneys. Accurate monitoring of the oxygenation status, however, is challenging due to the inaccessibility of the kidneys which are deep in the abdominal cavity in some patients. Near-Infrared spectroscopy (NIRS) measures regional oximetry, and has some utility in babies and slender adults in measuring oxygenation of the kidneys, but does not have the depth of penetration and specificity required for most adults. In addition, the depth penetration of NIRS limits the measurements to the cortical kidney tissue. Studies have shown that it is the medulla of the kidney may become hypoxic before the cortex and, in such cases, the hypoxia of the medulla may be a relatively early indicator of AKI (actual AKI or an increased risk of developing AKI).

It is believed that urine oxygen tension (uPO₂) may correlate with the oxygenation of the medulla. In examples described herein, a kidney monitoring system is configured to determine uPO₂ in urine output by a patient based on one or more sensed values, determine a volume of urine output (also referred to herein as the output of the urine) of the patient, and predict if the patient will develop AKI based on the determined uPO₂ and the determined output of the urine. In some examples, processing circuitry of the system is configured to determine the risk a patient may develop AKI (e.g., an AKI risk score indicative of the possibility of the patient developing AKI) based on the determined uPO₂ and the determined output of the urine. By determining the risk that a patient may develop AKI, the system may facilitate earlier intervention by a clinician to reduce the chance that the patient may develop AKI or reduce the severity of the AKI.

Risk of developing AKI may vary between patients, even patients having the same values of the parameters. Thus, it may be beneficial to determine one or more patient-specific baselines for a patient prior to determining the risk of the patient developing AKI. In some example systems described herein, processing circuitry of the system may relatively quickly estimate these patient-specific baselines and may use the patient-specific baselines to determine patient-specific thresholds against which the parameters may be compared when determining the risk the patient may develop AKI. Comparison of parameters against patient-specific thresholds may be more indicative of the risk a specific patient may develop AKI than comparison of parameters against general, predetermined thresholds.

In some examples, monitoring of changes in parameters may be more indicative of AKI than the parameters themselves due to patient-to-patient variability

In some examples, processing circuitry of the system may use the uPO₂ and output of the urine measurements to determine trends and/or total oxygen output which may also be used to determine the risk the patient may develop AKI. Therefore, in some examples, processing circuitry of the system may be configured to monitor trends of one or more parameters and determine an AKI risk score based at least in part on the trends of the one or more parameters.

The one or more sensors may be configured to generate signals indicative of a level of uPO₂ in urine (or other fluid) and the one or more sensors may be configured to generate signals indicative of a volume of urine output of a patient. These one or more sensors may be positioned at any suitable place, such as connected to a catheter (e.g., a Foley catheter) or otherwise in communication with fluid (e.g., urine) drained from the patient via the catheter.

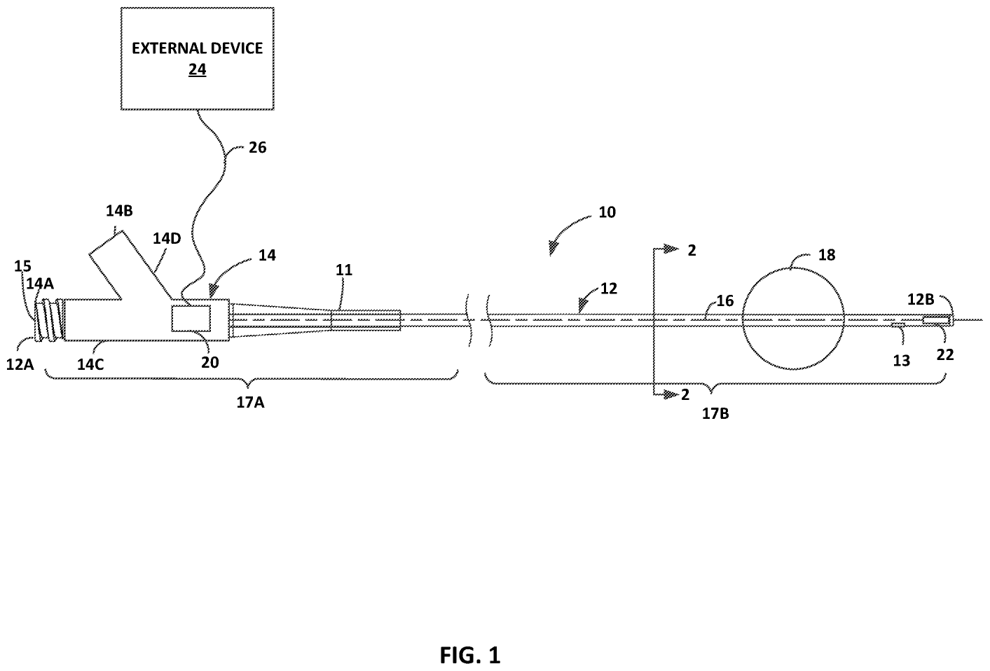

FIG. 1 is a conceptual side elevation view of an example medical device 10, which includes elongated body 12, hub 14, and anchoring member 18. In some examples, medical device 10 is a catheter, such as a Foley catheter. While a Foley catheter and its intended use is primarily referred to herein to describe medical device 10, in other examples, medical device 10 can be used for other purposes, such as to drain wounds or for intravascular monitoring or medical procedures.

Medical device 10 includes a distal portion 17A and a proximal portion 17B. Distal portion 17A includes a distal end 12A of elongated body 12 and is intended to be external to a patient's body when in use, while proximal portion 17B includes a proximal end 12B of elongated body 12 and is intended to be internal to a patient's body when in use. For example, when proximal portion 17B is positioned within a patient, e.g., such that proximal end 12B of elongated body 12 is within the patient's urethra and bladder, distal portion 17A may remain outside of the body of the patient.

As used herein, “sense” may include detect and/or measure. As used herein, “proximal” is used as defined in Section 3.1.4 of ASTM F623-19, Standard Performance Specification for Foley Catheter. That is, the proximal end of a catheter is the end closest to the patient when the catheter is being used by the patient. The distal end is therefore the end furthest from the patient.

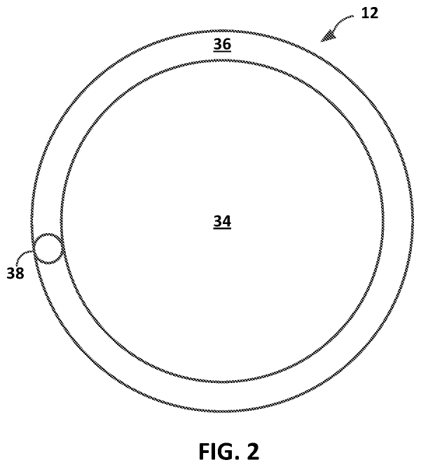

Elongated body 12 is a body that extends from distal end 12A to proximal end 12B and defines one or more inner lumens. In the example shown in FIGS. 1 and 2, elongated body 12 defines lumen 34 and lumen 36 (shown in FIG. 1). In some examples, lumen 34 may be a drainage lumen for draining a fluid from a target site, such as a bladder. In other examples, lumen 34 may be used for any other suitable purpose, such as to deliver a substance or another medical device to a target site within a patient. Lumen 34 may extend from fluid opening 13 to fluid opening 14A. Both fluid opening 13 and fluid opening 14A may be fluidically coupled to lumen 34, such that a fluid may flow from one of fluid opening 13 or fluid opening 14A to the other of fluid opening 13 or fluid opening 14A through lumen 34. In the example where lumen 34 is a drainage lumen, fluid opening 13 and fluid opening 14A may be drainage openings. In the example shown in FIG. 1, distal end 12A of elongated body 12 is received within hub 14 and is mechanically connected to hub 14 via an adhesive, welding, or another suitable technique or combination of techniques.

In some examples, elongated body 12 has a suitable length for accessing the bladder of a patient through the urethra. The length may be measured along central longitudinal axis 16 of elongated body 12. In some examples, elongated body 12 may have an outer diameter of about 12 French to about 14 French, but other dimensions may be used in other examples. Distal and proximal portions of elongated body 12 may each have any suitable length.

Hub 14 is positioned at a distal end of elongated body 12 and defines an opening through which the one or more inner lumens (e.g., lumen 34 shown in FIG. 2) of elongated body 12 may be accessed and, in some examples, closed. While hub 14 is shown in FIG. 1 as having two arms, 14C and 14D, (e.g., a “Y-hub”), hub 14 may have any suitable number of arms, which may depend on the number of inner lumens defined by elongated body 12. For example, each arm may be fluidically coupled to a respective inner lumen 34, 36 of elongated body 12. In the example of FIG. 1, hub 14 comprises a fluid opening 14A, which is fluidically coupled to lumen 34, and an inflation opening 14B, which is fluidically coupled to an inflation lumen 36 (shown in FIGS. 2A and 2B) of elongated body 12. In examples in which anchoring member 18 does not include an expandable balloon, rather than defining inflation lumen 36, elongated body 12 may define an inner lumen configured to receive a deployment mechanism (e.g., a pull wire or a push wire) for deploying an expandable structure anchoring member 18 and hub 14 may comprise fluid opening 14A and an opening 14B via which a clinician may access the deployment mechanism.

In examples in which medical device 10 is a Foley catheter, a fluid collection container (e.g., a urine bag) may be attached to fluid opening 14A for collecting urine draining from the patient's bladder. Inflation opening 14B may be operable to connect to an inflation device to inflate anchoring member 18 positioned on proximal portion 17B of medical device 10. Anchoring member 18 may be uninflated or undeployed when not in use. Hub 14 may include connectors, such as connector 15, for connecting to other devices, such as the fluid collection container and the inflation source. In some examples, medical device 10 includes strain relief member 11, which may be a part of hub 14 or may be separate from hub 14.

Proximal portion 17B of medical device 10 comprises anchoring member 18, fluid opening 13, and first sensor 22. While first sensor 22 is shown located in proximal portion 17B of medical device 10, first sensor 22 may be located anywhere on medical device 10 or distal to a distal end 12A of medical device 10. Anchoring member 18 may include any suitable structure configured to expand from a relatively low-profile state to an expanded state in which anchoring member 18 may engage with tissue of a patient (e.g., inside a bladder) to help secure and prevent movement of proximal portion 17B out of the body of the patient. For example, anchoring member 18 may include an anchor balloon or other expandable structure. When inflated or deployed, anchoring member 18 may function to anchor medical device 10 to the patient, for example, within the patient's bladder. In this manner, the portion of medical device 10 on the proximal side of anchoring member 18 may not slip out of the patient's bladder. Fluid opening 13 may be positioned on the surface of longitudinal axis of medical device 10 between anchoring member 18 and the proximal end 12B (as shown) or may be positioned at the proximal end 12B.

First sensors, e.g., first sensor 22, may be one or more sensors that are configured and intended to sense parameters that should be sensed relatively close to the fluid source, such as the bladder, because the parameters may substantially change as a function of time or based on the location at which the parameter is sensed. In some examples, first sensor 22 may include an oxygen sensor configured to sense uPO₂ in urine.

Temperature is one example parameter that may substantially change as a function of time and pressure is one example parameter that may change based on the location at which the parameter is sensed. Thus, temperature and pressure are two parameters that are better sensed at the proximal portion 17B of elongated body 12 (close to the fluid source), and, in some examples, first sensor 22 may comprise sensors such as a temperature sensor and/or pressure sensor. First sensor 22 may communicate sensor data to external device 24 via an electrical, optical, wireless or other connection. In some examples, first sensor 22 may communicate sensor data to external device 24 through a connection(s) within elongated body 12 of medical device 10 from proximal portion 17B to distal portion 17A via embedded wire(s) or optical cable(s). In other examples, first sensor 22 may communicate sensor data to external device 24 via a wireless communication technique.

Distal portion 17A of medical device 10 includes one or more second sensors 20. Second sensor 20 may be positioned on hub 14, as shown, or may be positioned elsewhere on distal portion 17A of the body of medical device 10, or may be positioned distal to distal end 12A, e.g., on tubing connected to a fluid collection container (e.g., a urine bag) or the like.

Second sensors, such as second sensor 20, may be sensors that are relatively larger, require relatively more electrical, optoelectrical and/or optical connections, and/or that sense parameters that may be sensed relatively far away from the fluid source compared to the parameters sensed by first sensor 22. Thus, the one or more parameters second sensor 20 are configured to sense may include parameters that do not substantially change as a function of time or based on the location at which the parameter is sensed. In some examples, the one or more parameters second sensor 20 may be configured to sense may include parameters that do substantially change as a function of time or based on the location at which the parameter is sensed. In some examples, second sensor 20 may include sensors configured to sense urine output (e.g., fluid flow or volume), urine concentration, amount of dissolved oxygen in the urine (oxygen tension or uPO₂), amount of dissolved carbon dioxide in the urine, urine pH, urine color, urine creatinine, and/or motion.

For example, elongated body 10 may be configured to reduce the amount of change in the amount of dissolved oxygen in the urine as the urine travels from fluid opening 13 to second sensor 20, in which case, second sensor 20 may include an oxygen sensor. For example, elongated body 10 may be configured as discussed in U.S. patent application Ser. No. 16/854,592, filed Apr. 21, 2020, and entitled “CATHETER INCLUDING A PLURALITY OF SENSORS.”

In some examples, first sensor 22 and/or second sensor 20 are mechanically connected to elongated body 12 or another part of medical device 10 using any suitable technique, such as, but not limited to, an adhesive, welding, by being embedded in elongated body 12, via a crimping band or another suitable attachment mechanism or combination of attachment mechanisms. In some examples, second sensor 20 is not mechanically connected to elongated body 12 or medical device 10, but is instead mechanically connected to a structure that is distal to distal end 12A of medical device 10, such as to tubing that extends between hub 14 and a fluid collection container.

First sensor 22 and second sensor 20 may be configured to communicate sensor data to an external device 24. External device 24 may be a computing device, such as a workstation, a desktop computer, a laptop computer, a smart phone, a tablet, a server or any other type of computing device that may be configured to receive, process and/or display sensor data. First sensor 22 and second sensor 20 may communicate sensor data to the external device via a connection 26. Connection 26 may be an electrical, optical, wireless or other connection.

Although only one first sensor 22 and only one second sensor 20 is shown in FIG. 1, in other examples, medical device 10 can include any suitable number of sensors on proximal portion 17B and any suitable number of sensors on distal portion 17A, where the sensors on proximal portion 17B sense the same or different parameters and the sensors on distal portion 17A sense the same or different parameters. In addition, some or all of the sensors on proximal portion 17B can sense the same or different parameters as the sensors on distal portion 17A. For example, in the case where sensors on the distal portion may be temperature dependent, it may be desirable to sense temperature both on the proximal portion 17B and the distal portion 17A.

Elongated body 12 may be structurally configured to be relatively flexible, pushable, and relatively kink- and buckle-resistant, so that it may resist buckling when a pushing force is applied to a relatively distal portion of the medical device to advance the elongated body proximally through the urethra and into the bladder. Kinking and/or buckling of elongated body 12 may hinder a clinician's efforts to push the elongated body proximally.

In some examples, at least a portion of an outer surface of elongated body 12 includes one or more coatings, such as an anti-microbial coating, and/or a lubricating coating. The lubricating coating may be configured to reduce static friction and/ kinetic friction between elongated body 12 and tissue of the patient as elongated body 12 is advanced through the urethra.

FIG. 2 is a diagram illustrating an example cross-section of medical device 10, where the cross-section is taken along line 2-2 in FIG. 1 in a direction orthogonal to central longitudinal axis 16. Anchoring member 18 is not shown in FIG. 2. FIG. 2 depicts a cross section of elongated body 12, which defines lumen 34 and lumen 36. In some examples, lumen 34 may be referred to as a drainage lumen, such as in examples in which medical device 10 is a Foley catheter configured to drain urine from a bladder of a patient, and lumen 36 may referred to as an inflation lumen in examples in which lumen 36 is configured to deliver an inflation fluid to anchoring member 18. Elongated body 12 may enclose connection 38. Lumen 34 may serve as a passage for urine entering medical device 10 through fluid opening 13 to fluid opening 14A.

Inflation lumen 36 may serve as a passage for a fluid, such as sterile water or saline, or a gas, such as air, from inflation opening 14B to anchoring member 18. For example, an inflation device (not shown) may pump fluid or gas into inflation lumen 36 through inflation opening 14B into anchoring member 18 such that anchoring member 18 is inflated to a size suitable to anchor medical device 10 to the patient's bladder. While inflation lumen 36 is shown as circular in cross section, it may be of any shape. In some examples, there may be a plurality of inflation lumens. For example, a plurality of inflation lumens may substantially surround lumen 34. In some examples, anchoring member 18 may be an expandable structure that is not an inflatable balloon. In such examples, inflation lumen 36 may be replaced by a deployment mechanism which may permit a clinician to expand the expandable structure or a lumen configured to house such a deployment mechanism. For example, inflation lumen may be replaced by a mechanical device that may be pushed and pulled separately from the medical device 10 by a clinician to expand or retract the expandable structure.

Connection 38 may serve to connect first sensor 22 positioned at proximal portion 17B to connection 26 (of FIG. 1). Connection 38 may be an electrical, optical or other connection. In some examples, connection 38 may comprise a plurality of connections. For example, connection 38 may include one of more wired or optical connections to a temperature sensor and one or more connections to a pressure sensor. In some examples, connection 38 may include one or more power connections to power first sensor 22 and one or more communications connections to receive sensor data from first sensor 22.

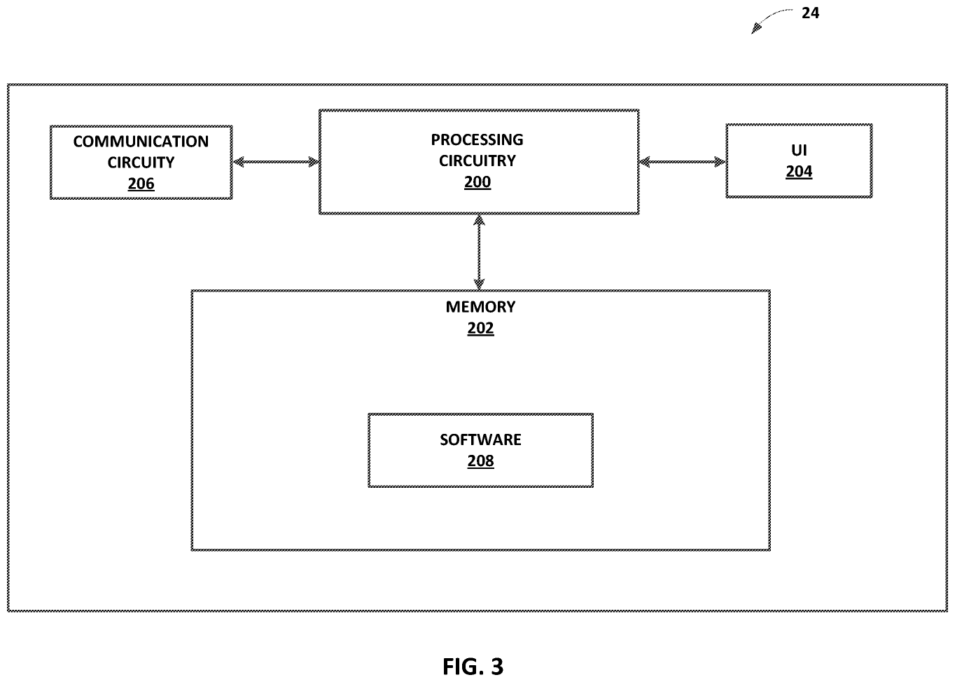

FIG. 3 is a functional block diagram illustrating an example external device 24 of FIG. 1. External device 24 may be configured to communicate with first sensor 22 and second sensor 20 and/or receive signals from first sensor 22 and second sensor 20. External device 24 may use a first signal from first sensor 22 and a second signal from second sensor 20 when determining a risk of a patient developing AKI (also referred to herein as a risk score or an AKI risk score). In the example of FIG. 3, external device 24 includes processing circuitry 200, memory 202, user interface (UI) 204, and communication circuitry 206. External device 24 may be a dedicated hardware device with dedicated software for the reading sensor data. Alternatively, external device 24 may be an off-the-shelf computing device, e.g., a desktop computer, a laptop computer, a tablet, or a smartphone running an application that enables external device 24 to read sensor data from first sensor 22 and second sensor 20 and determine an AKI risk score.

In some examples, a user of external device 24 may be a clinician. In some examples, a user uses external device 24 to monitor a patient's kidney function and to obtain an assessment of the risk a patient will develop AKI. In some examples, the user may interact with external device 24 via UI 204, which may include a display configured to present a graphical user interface to the user and/or sound generating circuitry configured to generate an audible output, and a keypad or another mechanism (such as a touch sensitive screen) for receiving input from the user. External device 24 may communicate with first sensor 22 and/or second sensor 20 using wired, wireless or optical methods through communication circuitry 206. In some examples, UI 204 may display a representation of the risk of the patient developing AKI, such as an AKI risk score. By displaying a representation of the risk of the patient developing AKI, external device 24 may inform a clinician of the risk that the patient develops AKI and facilitate earlier intervention by a clinician to reduce the chance that the patient may develop AKI or reduce the severity of the AKI

Processing circuitry 200 may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 200 may include multiple components, such as any combination of one or more microprocessors, one or more DSPs, one or more ASICs, or one or more FPGAs, as well as other discrete or integrated logic circuitry, and/or analog circuitry.

Memory 202 may store program instructions, such as software 208, which may include one or more program modules, which are executable by processing circuitry 200. When executed by processing circuitry 200, such program instructions may cause processing circuitry 200 and external device 24 to provide the functionality ascribed to them herein. The program instructions may be embodied in software and/or firmware. Memory 202 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

In some examples, processing circuitry 200 is configured to use an algorithm which may be stored in software 208 of memory 202 to determine a likelihood that a patient may develop AKI based on sensed uPO₂ and urine output, a first baseline of uPO₂, and a second baseline of total oxygen output in the urine. The sensed uPO₂ and output of urine parameters of the urine may be sensed via one or more sensors (e.g., sensors 20, 22 of FIG. 1) of the kidney function monitoring system. Processing circuitry 200 may determine the first threshold value and the second threshold value, as discussed further below, which may be patient-specific.

For example, external device 24 may monitor the partial pressure of oxygen in the urine (uPO₂) in the bladder, for example, via sensor 20 or sensor 22, as this measurement may reflect the oxygenation of the kidneys. In some examples, external device 24 may employ an algorithm (which may be stored in software 208 of memory 202) that takes the output of a sensor, such as sensor 20 or sensor 22, that measures an amount of oxygen dissolved in the urine (uPO₂) and a sensor, such as sensor 20 or sensor 22, that measures urine output (UO) to estimate the risk of developing AKI (e.g., through an AKI risk score). In some examples, processing circuitry 200 is configured to use an algorithm which may be stored in software 208 of memory 202 to determine trends and/or total oxygen output which may also be used to determine the AKI risk score.

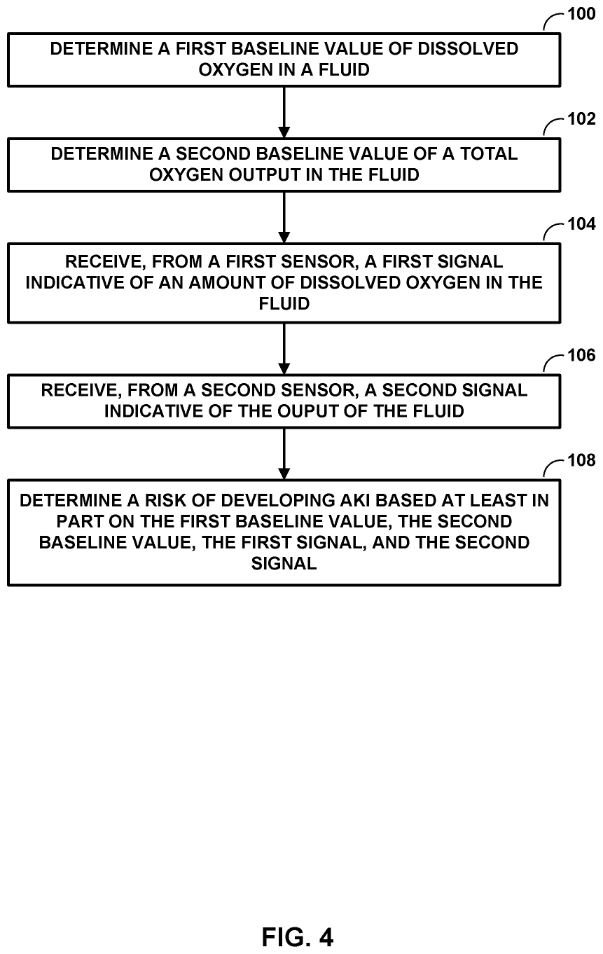

FIG. 4 is a flow diagram illustrating example techniques of determining an AKI risk score. While FIG. 4, as well as other techniques described herein, is described with reference to processing circuitry 200 of external device 24, in other examples, other processing circuitry alone or in combination with processing circuitry 200 may perform all or part of the techniques described herein. The other processing circuitry may, for example, be remotely located from the patient and/or external device 24, such as at a relatively central location that receives and processes data from multiple patients.

In accordance with the technique shown in FIG. 4, processing circuitry 200 determines a first baseline value of dissolved oxygen in a fluid (100). For example, processing circuitry 200 may determine at least two measures of the amount of dissolved oxygen in the fluid based on a first signal and apply at least one of an exponential decay or a non-linear regression to the at least two measures of the amount of dissolved oxygen in the fluid when determining the first baseline value of dissolved oxygen in the fluid. In some examples, processing circuitry 200 may read the first baseline from memory, such as memory 202, or memory of another device. In some examples, the fluid is urine and the urine is output from a bladder of a patient.

In some examples, the first signal may be a signal from a first sensor (e.g., first sensor 22 or second sensor 20 of FIG. 1). In some examples, the first sensor may be a dissolved oxygen sensor. Measures (e.g., quantitative values) of dissolved oxygen in a fluid, such as urine, may change over time. Processing circuitry 200 may determine, based on the first signal, a first measure of dissolved oxygen in the fluid that is below a predetermined threshold value and average, the first measure and measures of the dissolved oxygen in the fluid prior to the first measure when determining the first baseline value of dissolved oxygen in the fluid. For example, processing circuitry 200 may average the first measure of dissolved oxygen in the fluid that is below the predetermined threshold with all prior measures of dissolved oxygen in the fluid, with a predetermined number of prior measures of dissolved oxygen in the fluid (e.g., as low as 0 measures if the predetermined threshold is input by a user, or as few as 3 or more relatively stable measures if the predetermined threshold is not input by the user, depending on the sampling rate), or with prior measures of dissolved oxygen in the fluid within a predetermined time period immediately prior to the first measure (e.g., as few as 10-15 minutes up to 60 minutes or longer).

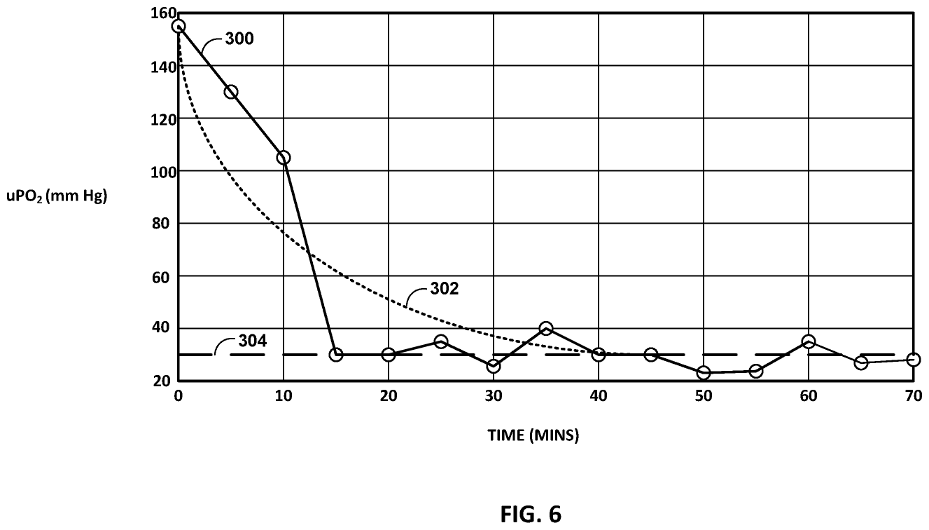

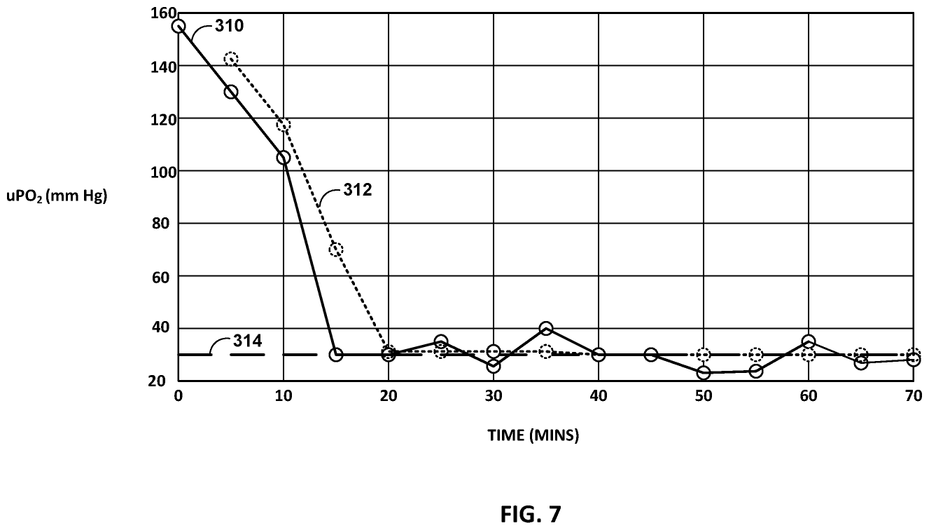

Cardiopulmonary bypass surgery can create relatively large changes in the signals that change as a function of urine oxygen tension and fluid output, and the period just before it begins may be a valid time period to be considered as the patient's baseline. Further, a bypass machine may create a higher uPO₂ baseline, which may not be as conducive to determining an AKI risk score. Therefore, in another example, processing circuitry 200 may average measures of the dissolved oxygen during a time period immediately before cardiopulmonary bypass surgery occurring to a patient when determining the first baseline value of dissolved oxygen in the fluid. The first baseline is discussed in more detail below with respect to FIGS. 6 and 7.

Processing circuitry 200 determines a second baseline value of a total oxygen output in the fluid (102). For example, processing circuitry 200 may determine at least two measures of the amount of dissolved oxygen in the fluid based on the first signal and determine at least two measures of the output of the fluid based on a second signal. In some examples the second signal may be from a second sensor (e.g., sensor 20 or sensor 22 of FIG. 1). In some examples, the second sensor may be a volume sensor or a flow sensor. Processing circuitry 200 may also apply at least one of an exponential decay or a non-linear regression to at least one of: a) the at least two measures of the amount of dissolved oxygen in the fluid; b) the at least two measures of the output of the fluid based on the second signal; or c) at least two measures of the total oxygen output in the fluid based on the at least two measures of the dissolved oxygen in the fluid and the at least two measures of the amount of dissolved oxygen in the fluid, when determining the second baseline value of total oxygen output in the fluid. In some examples, the processing circuitry 200 may read the second baseline from memory, such as memory 202 or memory of another device. The second baseline is discussed in more detail below with respect to FIGS. 6 and 7.

Processing circuitry 200 receives, from the first sensor, a first signal indicative of an amount of dissolved oxygen in the fluid (104). For example, processing circuitry 200 may receive the first signal from a dissolved oxygen sensor.

Processing circuitry 200 may receive, from the second sensor, a second signal indicative of the output of the fluid (106). For example, processing circuitry 200 may receive the second signal from a volume sensor or flow sensor.

Processing circuitry 200 determines a risk of developing AKI based at least in part on the first baseline value, the second baseline value, the first signal, and the second signal (108). By determining the risk that a patient may develop AKI, the system may facilitate earlier intervention by a clinician to reduce the chance that the patient may develop AKI or reduce the severity of the AKI

For example, as part of determining the AKI risk score, processing circuitry 200 may determine the amount of dissolved oxygen in the fluid based on the first signal and compare the amount of dissolved oxygen in the fluid to a first threshold value. Processing circuitry 200 may determine the output of the fluid based on the second signal and compare the output of the fluid to a second threshold value. Processing circuitry 200 may compare the measure of total oxygen output to a third threshold value. In these examples, processing circuitry 200 can determine the risk of developing AKI based on the comparisons. In some examples, the first threshold value is based on the first baseline value and the second threshold value is based on the second baseline value. The first, second, and third threshold values, as well as other thresholds described herein, can be stored by memory 202 of external device 24 or a memory of another device.

In some examples, processing circuitry 200 may determine a measure of total oxygen output in the fluid based on the first signal and the second signal, and determine the risk of developing AKI based on the measure of total oxygen output, alone or in combination with the comparisons discussed above.

In some examples, as part of determining the risk of developing AKI, processing circuitry 200 may determine, based on the first signal, a first trend in the amount of dissolved oxygen in the fluid over time. Processing circuitry 200 may also determine, based on the first signal and the second signal, a second trend in the measure of total oxygen output in the fluid. Processing circuitry 200 may also compare the first trend to a fourth threshold value and compare the second trend to a fifth threshold value. In these examples, determining the risk of developing AKI may be based on the comparisons. For example, processing circuitry 200 can determine the risk of developing AKI based on an amount of time (“first” amount of time) the first trend is below the fourth threshold value and an amount of time (“second” amount of time) the second trend is below the fifth threshold value, alone or in combination with the comparisons using the first, second, and third threshold values discussed above.

In some examples, as part of determining the risk of developing AKI, processing circuitry 200 may determine, based on the second signal, a third trend in the output of the fluid over time and compare the third trend to a sixth threshold value. In these examples, determining the risk of developing AKI may be based on the comparison. For example, processing circuitry 200 can determine the risk of developing AKI is based on an amount of time (“third” amount of time) the third trend is below the sixth threshold value, alone or in combination with the comparisons using the first, second, third, fourth and fifth threshold values discussed above.

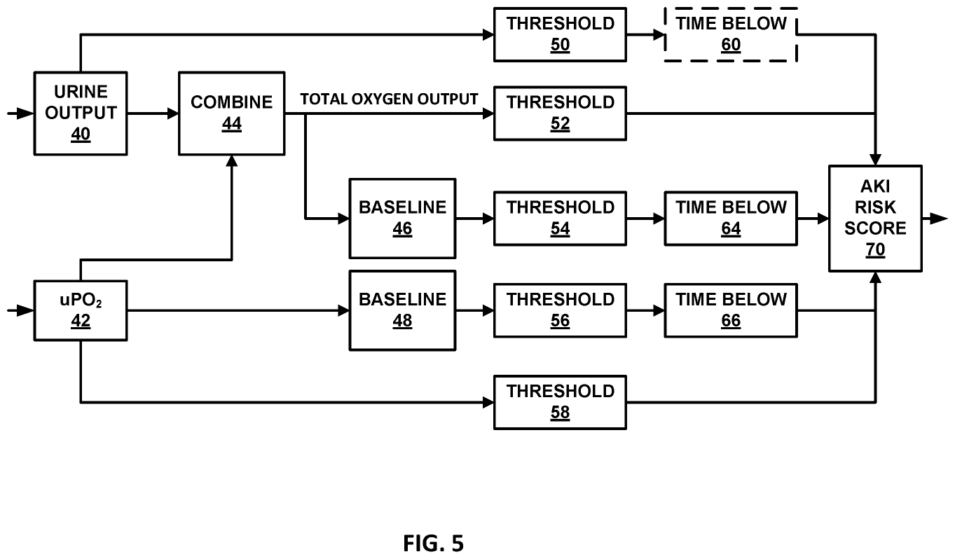

FIG. 5 is a flow diagram illustrating example techniques that processing circuitry 200 of external device 24 may implement to determine a risk score for a patient to develop AKI based on uPO₂ and/or urine output. FIG. 5 illustrates examples of all or part of the techniques of FIG. 4. For example, using the techniques of FIG. 5, processing circuitry 200 may determine the risk that a patient may develop AKI based on received measure of uPO₂ and urine output from first sensor 22 and/or second sensor 20. FIG. 5 may be representative of an algorithm stored in software 208 of memory 202 and executed by processing circuitry 200 (FIG. 3). While the techniques are described with reference to processing circuitry 200 of external device 24, in other examples, processing circuitry of another device alone or in combination with processing circuitry 200 may perform any portion of the techniques of this disclosure.

Processing circuitry 200 may receive signals indicative of a volume of urine output 40 and uPO₂ 42 from any suitable sensor configured to sense the respective parameter of urine or other fluid of a patient. For example, processing circuitry 200 may receive signals indicative of the sensed parameters from first sensor 22 and/or second sensor 20 (of FIG. 1). Processing circuitry 200 determines a patient-specific baseline 48 (the first baseline of FIG. 4) for uPO₂ and determines a patient-specific baseline 46 (the second baseline of FIG. 4) for total oxygen output. Example techniques for determining these baselines is discussed in further detail with respect to FIGS. 6 and 7. In some examples, processing circuitry 200 may combine the sensed urine output 40 with the sensed uPO₂ 42 in combine box 44 to determine a total oxygen output. For example, processing circuitry 200 may mathematically combine (such as using multiplication) sensed uPO₂ 42 with sensed urine output 40 to determine a measure of total oxygen output.

Processing circuitry 200 may determine a threshold 50 for urine output, a threshold 52 for total oxygen output, a threshold 54 for total oxygen output relative to the baseline, a threshold 56 for uPO₂ based on baseline, and/or set a threshold 58 for uPO₂ based on baseline 48. In some examples, processing circuitry 200 may set thresholds 50, 52, 54, 56, and/or 58. In some examples, processing circuitry 200 may read thresholds 50, 52, 54, 56, and/or 58 from memory, such as memory 202 or memory in a separate device. Thresholds 50, 52, 54, 56, and 58 can be numerical values in some examples. In some examples, thresholds 52 and 54 may be the same. In some examples, thresholds 52 and 54 may be different. In some examples, thresholds 56 and 58 may be the same. In some examples, thresholds 56 and 58 may be different. In some examples, threshold 52 may be based on threshold 54, or vice versa. In some examples, threshold 56 may be based on threshold 58, or vice versa. In some examples, threshold 52 may be based on baseline 46 and/or threshold 58 may be based on baseline 48. In some examples, thresholds based on baseline 46 and/or baseline 48 may be lower than baseline 46 and/or baseline 48, respectively. In some examples, thresholds based on baseline 46 and/or baseline 48 may be lower than the respective baseline 46, 48 by 5% to 50% of the baseline value, such as 20% less than baseline 46 and/or baseline 48, respectively.

Low urine output may be indicative of a patient not producing enough urine which may be indicative of a degradation of kidney function. Low uPO₂ and low total oxygen output in the urine may correlate to perfusion and/or oxygenation of the kidneys may be indicative of a degradation of kidney function. When the values of the parameters (urine output 40, uPO₂ 42, total oxygen output) of FIG. 5 are below the respective thresholds, it may be more likely that a patient may develop AKI. Therefore, processing circuitry 200 may compare measures of one or more the sensed parameters to the respective thresholds to determine an AKI risk score 70. Such comparison(s) may be performed, for example, before a medical procedure, during the medical procedure and/or during recovery from the medical procedure. For example, processing circuitry 200 may compare the urine output 40 to threshold 50 and determinate AKI risk score 70 based on the results of the comparison. In some examples, alternatively, or additionally, processing circuitry 200 may compare a urine output trend over time to threshold 50 and determine an amount of time the urine output is below 60 threshold 50 and determine the AKI risk score 70 based on the determined amount of time.

In addition to the aforementioned comparisons, in some examples, processing circuitry 200 may compare the total oxygen output to threshold 52 and determine the AKI risk score 70 based on the comparison and/or compare the total oxygen output trend over time to threshold 54 and determine an amount of time below 64 threshold 54 and determine the AKI risk score 70 based on the determined amount of time. In some examples, in addition to, or instead of the aforementioned comparisons, processing circuitry 200 may compare the uPO₂ trend over time to threshold 56 and determine an amount of time the uPO₂ is below 66 threshold 56 and determine the AKI risk score 70 based on the determined amount of time. As another example of a threshold comparison, in addition to, or instead of any of the aforementioned examples, processing circuitry 200 may compare the uPO₂ to threshold 58 and determine the AKI risk score 70 based on the comparison.

By utilizing trends over time (e.g., changes in total oxygen output over time and/or changes in uPO₂ over time), processing circuitry 200 may correct for changes in urine flow which could cause the uPO₂ to increase or decrease in a way that may not be reflective of the actual kidney oxygenation. Hence, from the two sensed inputs, processing circuitry 200 of external device 24 may determine at least five parameters: uPO₂, a uPO₂ trend, urine output (and/or urine output trend), total oxygen output, and a total oxygen output trend which processing circuitry 200 may use to determine the risk that a patient may develop AKI.

In some examples, based on a consideration of one or more of the parameters of FIG. 5, and/or the times below 60, 64, and/or 66 a threshold (or, in some examples, an area under the curve relative to a baseline), processing circuitry 200 may determine and output AKI risk score 70 via user interface 204. AKI risk score 70 can be quantitative or qualitative in various examples. For example, AKI risk score 70 may be a value in a continuous index, such as a numeric range between 1-10 or 1-100, a percentage value of kidney function or risk level, a discrete index, such as low, medium, high, or a qualitative indication, such as using a color scale (e.g., red indicates a relatively high risk of developing AKI and green indicates a relatively low risk). In some examples, processing circuitry 200 may take into account observed data in clinical trials, animal studies, or both clinical trials and animal studies when determining AKI risk score 70 may. In some examples, processing circuitry 200 may utilize an algebraic formula or leverage machine learning (e.g., via a neural network) and utilize a more complicated algorithm than a simple regression model when determining AKI risk score 70.

In some examples, AKI risk score 70 may be based on combining the parameters (urine output 40, uPO₂ 42, total oxygen output) or the results of the comparisons to the thresholds 50-58. In some examples, processing circuitry 200 may determine AKI risk score 70 based on less than five parameters. For example, processing circuitry 200 may determine AKI risk score 70 based on the absolute measure of two parameters, the interaction between two parameters, or the relative change in the parameters, or the interaction of the relative change in the parameters. In some examples, additional parameters, such as a sensed temperature, may be used in a similar manner. In some examples, the parameters may be input to a neural network (e.g., using machine learning) to determine AKI risk score 70. In some examples, processing circuitry 200 may use a look-up table to determine AKI risk score 70 based upon the parameters and determinations. In some examples, an algorithm may be used to compare measured parameter values to historic data, which may be patient-specific or anonymized historic data of other patients, or both.

Baselines 46, 48 may be determined using any suitable technique. On initial insertion of a catheter into a bladder of a patient, there is an initial period of time that a urine flow rate and urine oxygenation (uPO₂) may decrease from relatively high values. For example, if the bladder is relatively full, then there may a relatively high volume of fluid out of the bladder via the catheter until the catheter drains the bladder in more real time. As another example, uPO₂ may be relatively high upon initial introduction of the catheter into the bladder if patients may have been breathing supplemental oxygenation concentrations of around 60% before surgery starts. Because the patient may be breathing around 60% oxygen, the partial pressure of oxygen in arterial blood (PaO₂) may be elevated, such as in the range of 200 mmHg. As the urine has been accumulating in the bladder, the volume of urine increases and the urine oxygenation starts to equilibrate to the surrounding tissue, which may have a high oxygen content (−200 mmHg). Hence, when medical device 10 is initially inserted into the bladder of a patient, the urine drains at a high rate with a high oxygen content that may not be informative of the oxygenation state of the patient's kidneys. However, after a period of time, the flow rate and oxygen levels of the urine stabilize to values that may be clinically relevant and may provide important diagnostic information to the clinician.

In some examples, processing circuitry 200 of external device 24 may receive from first sensor 22 and/or second sensor 20 signals indicative of urine volume (e.g., such as volume itself or flow rate) and/or oxygenation (e.g., uPO₂) and may determine initial changes in urine volume and/or oxygenation to provide an estimation of the baseline of volume or flow rate and/or oxygen levels (e.g., uPO₂ and/or total oxygen output). Processing circuitry 200 may periodically or continually the respective parameter level and update the estimated baseline value until a relatively high level of confidence of accurate baseline volume (e.g., volume or flow) and oxygen levels are established.

As discussed above, risk of developing AKI may vary between patients, even patients having the same values of the parameters. As such, a generic baseline values used across all patients may be less accurate than patient-specific baseline values. Additionally, determining the patient-specific baseline values as quickly as possible may reduce the time with which an accurate risk assessment may be made. Therefore, it may be beneficial to quickly determine a patient-specific baseline value, e.g., of uPO₂ of the urine, the total oxygen output in the urine and/or the urine volume or flow, with which processing circuitry 200 can determine AKI risk score 70. For example, urine exiting the kidneys and entering the bladder may become acclimated to the bladder relatively quickly. For example, the first few (e.g., three) measurements of uPO₂ may be more indicative of a bladder wall than of urine in the kidneys. In addition, sensed urine volume or flow (which may be used together with uPO₂ to determine total oxygen output in the urine) may be relatively large or heavy when a catheter is first inserted into the patient than at other times. Thus, these initial values may not be an accurate representation of uPO₂ or urine volume or flow. However, processing circuitry 200 may use these initial values to predict or establish patient-specific baseline(s) and use the patient-specific baseline(s) to determine thresholds against which processing circuitry 200 may compare parameters and trends of the parameters to determine the risk of a patient developing AKI.

FIG. 6 is a graph illustrating example measurements of uPO₂ over time, modeled initial decrease in uPO₂, and baseline uPO₂. In the example of FIG. 6, sensed uPO₂ values from a patient are represented by the circles along solid line 300. Solid line 300 itself represents an approximation of the sensed values at the times between the measurements. Dashed line 304 represents a baseline value uPO₂ of approximately 30 mmHg which may be a patient-specific baseline. Dotted line 302 represents a simple exponential decay that processing circuitry 200 may use to model the initial decrease of the uPO₂ values in the urine of the patient.

This example shows the how the uPO₂ value of the urine of the patient starts at a high value ˜160 mmHg and decreases to a baseline value around 30 mmHg. The solid line 300 represents the sensed uPO₂ with time zero corresponding to the initial insertion of the proximal end of the Foley catheter into the bladder of the patient.

In some examples, processing circuitry 200 of external device 24 may use a simple exponential decay to model the initial decrease such as that of dotted line 302. From FIG. 6, using a simple exponential decay, the dotted line 302 may be used to determine baseline value 304 with a reasonable approximation in a 30-40 minute time frame. However, when using a non-linear regression, data points up to 60 minutes may be needed to determine baseline value 304. For example, processing circuitry 200 may determine at least two measures of the amount of dissolved oxygen in the fluid based on a first signal and apply at least one of an exponential decay or a non-linear regression to the at least two measures of the amount of dissolved oxygen in the fluid when determining the first baseline value of dissolved oxygen in the fluid.

Similarly, to establish the baseline value uPO₂, processing circuitry 200 may establish a second baseline value of total oxygen output. For example, processing circuitry 200 may use a simple exponential decay or a non-linear regression of the at least two measures of the uPO₂ (the first baseline) and a simple exponential decay or a non-linear regression of at least two corresponding measures of the output of the fluid (a baseline of the output of the fluid) to determine the second baseline value of total oxygen output. For example, processing circuitry 200 may mathematically combine the first baseline and the baseline of the output of the fluid. Alternatively, processing circuitry 200 may mathematically combine at least two measures of the amount of dissolved oxygen in the fluid and at least two corresponding measures of the output of the fluid to determine at least two measures of total oxygen output and may use a simple exponential decay or a non-linear regression of the at least two measures of the total oxygen output to determine the second baseline.

If processing circuitry 200 uses more complicated baseline prediction algorithms, then the time to estimate the baseline uPO₂ may decrease. For example, if the rate of change of the uPO₂ is incorporated into the estimation, then processing circuitry 200 may estimate the baseline uPO₂ significantly quicker, as shown in FIG. 7. FIG. 7 is a graph illustrating example measurements of uPO₂ over time, modeled initial decrease in uPO₂, and baseline uPO₂. Sensed uPO₂ values from a patient are represented by the circles along solid line 310. Solid line 310 represents an approximation of the sensed values at the times between the circled measurements. Dashed line 314 represents a baseline value uPO₂ of approximately 30 mmHg which may be patient-specific. Dotted line 312 represents a model processing circuitry 200 may use to estimate the baseline uPO₂ with dotted circles representing the modeled values of uPO₂ at times coinciding with actual measurements. It may be advantageous to establish a baseline measurement as quickly as possible and before cardiopulmonary bypass or other major maneuvers occur and cause a change to the baseline value.

In the example of FIG. 7, once the rate of change in uPO₂ is below a predetermined threshold value (e.g., 15 mmHg), processing circuitry 200 may store that time point in memory 202 as a stable point and may determine the baseline uPO₂ by at least averaging the data back to the initial time point (e.g., insertion of the proximal end of the catheter into the bladder of the patient). In this example, processing circuitry 200 may determine the baseline uPO₂ periodically (e.g., every 1-10 minutes, such as every 5 minutes) and processing circuitry 200 may determine a convergence to the patient baseline in, for example, 20 minutes. For example, processing circuitry 200 may determine, based on the first signal, a first measure of dissolved oxygen in the fluid that is below the predetermined threshold value and average the first measure and measures of the dissolved oxygen in the fluid prior to the first measure when determining the first baseline value of dissolved oxygen in the fluid.

Similarly, processing circuitry 200 may determine the second baseline by at least mathematically combining the first baseline and a baseline of the output of the fluid, or at least mathematically combining at least two measures of dissolved oxygen in the fluid and at least two corresponding measures of the output of the fluid.

In another example, processing circuitry 200 may determine a baseline measurement by at least retrospectively averaging the period directly before cardiopulmonary bypass begins. Cardiopulmonary bypass surgery can create relatively large changes in the signals that change as a function of urine oxygen tension and fluid output. Additionally, a cardiopulmonary bypass machine may create a higher uPO₂ in the urine of a patient which may bias a baseline. Therefore, the period just before cardiopulmonary bypass surgery begins may be a valid time period to be considered as the patient's baseline. For example, processing circuitry 200 may average measures of the dissolved oxygen during a time period immediately before cardiopulmonary bypass surgery occurring to a patient when determining the first baseline value of dissolved oxygen in the fluid.

The devices, systems, and techniques of this disclosure may determine a risk of a patient developing AKI. By determining the risk that a patient may develop AKI, the devices, systems, and techniques of this disclosure may facilitate earlier intervention by a clinician to reduce the chance that the patient may develop AKI or reduce the severity of the AKI.

The techniques described in this disclosure, including those attributed to sensor 20, sensor 22, processing circuitry 200, communication circuitry 206, and UI 204 or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

This disclosure includes the following non-limiting examples.

Example 1. A method comprising: determining, by processing circuitry, a first baseline value of dissolved oxygen in a fluid; determining, by the processing circuitry, a second baseline value of a total oxygen output in the fluid; receiving, from a first sensor, a first signal indicative of an amount of dissolved oxygen in the fluid; receiving, from a second sensor, a second signal indicative of the output of the fluid; and determining, by the processing circuitry, a risk of developing acute kidney injury (AKI) based at least in part on the first baseline value, the second baseline value, the first signal, and the second signal.

Example 2. The method of example 1, further comprising: determining, by the processing circuitry, a measure of total oxygen output in the fluid based on the first signal and the second signal, wherein determining the risk of developing AKI further comprises determining the risk of developing AKI based on the measure of total oxygen output.

Example 3. The method of example 2, wherein determining the risk of developing AKI comprises: determining, by the processing circuitry, the amount of dissolved oxygen in the fluid based on the first signal; comparing, by the processing circuitry, the amount of dissolved oxygen in the fluid to a first threshold value; determining, by the processing circuitry, the output of the fluid based on the second signal; comparing, by the processing circuitry, the output of the fluid to a second threshold value; and comparing, by the processing circuitry, the measure of total oxygen output to a third threshold value, wherein the risk of developing AKI is based on the comparisons.

Example 4. The method of example 3, wherein determining the risk of developing AKI comprises: determining, by the processing circuitry and based on the first signal, a first trend in the amount of dissolved oxygen in the fluid over time; determining, by the processing circuitry and based on the first signal and the second signal, a second trend in the measure of total oxygen output in the fluid; comparing, by the processing circuitry, the first trend to a fourth threshold value; and comparing, by the processing circuitry, the second trend to a fifth threshold value, wherein determining the risk of developing AKI is based on the comparisons.

Example 5. The method of example 4, wherein determining the risk of developing AKI is further based on a first amount of time the first trend is below the fourth threshold value and an amount of time the second trend is below the fifth threshold value.

Example 6. The method of example 4 or example 5, wherein determining the risk of developing AKI comprises: determining, by the processing circuitry and based on the second signal, a third trend in the output of the fluid over time; and comparing, by the processing circuitry, the third trend to a sixth threshold value, wherein determining the risk of developing AKI is based on the comparison.

Example 7. The method of example 6, wherein determining the risk of developing AKI is further based on a third amount of time the third trend is below the sixth threshold value.

Example 8. The method of any combination of examples 3-7, wherein the first threshold value is based on the first baseline value and the second threshold value is based on the second baseline value.

Example 9. The method of any combination of examples 3-8, wherein determining the first baseline value comprises: determining, by the processing circuitry and based on the first signal, a first measure of dissolved oxygen in the fluid that is below a predetermined threshold value; and averaging, by the processing circuitry, the first measure and measures of the dissolved oxygen in the fluid prior to the first measure.

Example 10. The method of any combination of examples 1-9, wherein determining the first baseline value comprises: averaging, by the processing circuitry, measures of the dissolved oxygen during a time period immediately before cardiopulmonary bypass surgery occurring to a patient.

Example 11. The method of any combination of examples 1-9, wherein determining the first baseline value comprises: determining, by the processing circuitry, at least two measures of the amount of dissolved oxygen in the fluid based on the first signal; and applying, by the processing circuitry, at least one of an exponential decay or a non-linear regression to the at least two measures of the amount of dissolved oxygen in the fluid.

Example 12. The method of any combination of examples 1-11, wherein determining the second baseline value comprises: determining, by the processing circuitry, at least two measures of the amount of dissolved oxygen in the fluid based on the first signal; determining, by the processing circuitry, at least two measures of the output of the fluid based on the second signal; and applying, by the processing circuitry, at least one of an exponential decay or a non-linear regression to at least one of: a) the at least two measures of the amount of dissolved oxygen in the fluid based on the first signal; b) the at least two measures of the output of the fluid based on the second signal; or c) at least two measures of the total oxygen output in the fluid based on the at least two measures of the dissolved oxygen in the fluid and the at least two measures of the amount of dissolved oxygen in the fluid.

Example 13. The method of any combination of examples 1-12, wherein the fluid is urine and the urine is output from a bladder of a patient.

Example 14. A device comprising: memory; and processing circuitry communicatively coupled to the memory, the processing circuitry being configured to: determine a first baseline value of dissolved oxygen in a fluid; determine a second baseline value of a total oxygen output in the fluid; receive, from a first sensor, a first signal indicative of an amount of dissolved oxygen in the fluid; receive, from a second sensor, a second signal indicative of the output of the fluid; and determine a risk of developing acute kidney injury (AKI) based at least in part on the first baseline value, the second baseline value, the first signal, and the second signal.

Example 15. The device of example 14, wherein the processing circuitry is further configured to: determine a measure of total oxygen output in the fluid based on the first signal and the second signal, wherein determining the risk of developing AKI further comprises determining the risk of developing AKI based on the measure of total oxygen output.

Example 16. The device of example 15, wherein as part of determining the risk of developing AKI, the processing circuitry is configured to: determine the amount of dissolved oxygen in the fluid based on the first signal; compare the amount of dissolved oxygen in the fluid to a first threshold value; determine the output of the fluid based on the second signal; compare the output of the fluid to a second threshold value; and compare the measure of total oxygen output to a third threshold value, wherein the risk of developing AKI is based on the comparisons.

Example 17. The device of example 16, wherein as part of determining the risk of developing AKI, the processing circuitry is configured to: determine, based on the first signal, a first trend in the amount of dissolved oxygen in the fluid over time; determine, based on the first signal and the second signal, a second trend in the measure of total oxygen output in the fluid; compare the first trend to a fourth threshold value; and compare the second trend to a fifth threshold value, wherein determining the risk of developing AKI is based on the comparisons.

Example 18. The device of example 17, wherein the processing circuitry is configured to determine the risk of developing AKI further based on a first amount of time the first trend is below the fourth threshold value and an amount of time the second trend is below the fifth threshold value.

Example 19. The device of example 17 or example 18, wherein as part of determining the risk of developing AKI, the processing circuitry is configured to: determine, based on the second signal, a third trend in the output of the fluid over time; and compare the third trend to a sixth threshold value, wherein determining the risk of developing AKI is based on the comparison.

Example 20. The device of example 19, wherein determining the risk of developing AKI is further based on a third amount of time the third trend is below the sixth threshold value.

Example 21. The device of any combination of examples 16-20, wherein the first threshold value is based on the first baseline value and the second threshold value is based on the second baseline value.

Example 22. The device of any combination of examples 16-21 wherein as part of determining the first baseline value, the processing circuitry is configured to: determine, based on the first signal, a first measure of dissolved oxygen in the fluid that is below a predetermined threshold value; and average the first measure and measures of the dissolved oxygen in the fluid prior to the first measure.

Example 23. The device of any combination of examples 14-22, wherein as part of determining the first baseline value, the processing circuitry is configured to: average measures of the dissolved oxygen during a time period immediately before cardiopulmonary bypass surgery occurring to a patient.

Example 24. The device of any combination of examples 14-23, wherein as part of determining the first baseline value, the processing circuitry is configured to: determine at least two measures of the amount of dissolved oxygen in the fluid based on the first signal; and apply at least one of an exponential decay or a non-linear regression to the at least two measures of the amount of dissolved oxygen in the fluid.

Example 25. The device of any combination of examples 14-24, wherein as part of determining the second baseline value, the processing circuitry is configured to: determine at least two measures of the amount of dissolved oxygen in the fluid based on the first signal; determine at least two measures of the output of the fluid based on the second signal; and apply at least one of an exponential decay or a non-linear regression to at least one of: a) the at least two measures of the amount of dissolved oxygen in the fluid; b) the at least two measures of the output of the fluid based on the second signal; or c) at least two measures of the total oxygen output in the fluid based on the at least two measures of the dissolved oxygen in the fluid and the at least two measures of the amount of dissolved oxygen in the fluid.

Example 26. The device of any combination of examples 14-25, wherein the fluid is urine and the urine is output from a bladder of a patient.

Example 27. A device comprising: memory; and processing circuitry communicatively coupled to the memory, the processing circuitry being configured to: determine at least two measures of an amount of dissolved oxygen in a fluid based on a first signal; apply, to determine a first baseline value of dissolved oxygen in the fluid, at least one of an exponential decay or a non-linear regression to the at least two measures of the amount of dissolved oxygen in the fluid; determine at least two measures of the output of the fluid based on a second signal; apply, to determine a second baseline value of a total oxygen output in the fluid, at least one of an exponential decay or a non-linear regression to at least one of: a) the at least two measures of the amount of dissolved oxygen in the fluid; b) the at least two measures of the output of the fluid based on the second signal; or c) at least two measures of the total oxygen output in the fluid based on the at least two measures of the dissolved oxygen in the fluid and the at least two measures of the amount of dissolved oxygen in the fluid; and determine a risk of developing acute kidney injury (AKI) based at least in part on the first baseline value, the second baseline value, the first signal, and the second signal.

Various examples have been described. These and other examples are within the scope of the following claims.

What is claimed is:

1. A method comprising:
determining, by processing circuitry, a first baseline value of dissolved oxygen in a fluid;
determining, by the processing circuitry, a second baseline value of a total oxygen output in the fluid;
receiving, from a first sensor, a first signal indicative of an amount of dissolved oxygen in the fluid;
receiving, from a second sensor, a second signal indicative of the output of the fluid; and
determining, by the processing circuitry, a risk of developing acute kidney injury (AKI) based at least in part on the first baseline value, the second baseline value, the first signal, and the second signal.

2. The method of claim 1, further comprising:
determining, by the processing circuitry, a measure of total oxygen output in the fluid based on the first signal and the second signal, wherein determining the risk of developing AKI further comprises determining the risk of developing AKI based on the measure of total oxygen output.

3. The method of claim 2, wherein determining the risk of developing AKI comprises:
determining, by the processing circuitry, the amount of dissolved oxygen in the fluid based on the first signal;
comparing, by the processing circuitry, the amount of dissolved oxygen in the fluid to a first threshold value;
determining, by the processing circuitry, the output of the fluid based on the second signal;
comparing, by the processing circuitry, the output of the fluid to a second threshold value; and
comparing, by the processing circuitry, the measure of total oxygen output to a third threshold value,
wherein the risk of developing AKI is based on the comparisons.

4. The method of claim 3, wherein determining the risk of developing AKI comprises:
determining, by the processing circuitry and based on the first signal, a first trend in the amount of dissolved oxygen in the fluid over time;
determining, by the processing circuitry and based on the first signal and the second signal, a second trend in the measure of total oxygen output in the fluid;
comparing, by the processing circuitry, the first trend to a fourth threshold value; and
comparing, by the processing circuitry, the second trend to a fifth threshold value,
wherein determining the risk of developing AKI is based on the comparisons.

5. The method of claim 4, wherein determining the risk of developing AKI is further based on a first amount of time the first trend is below the fourth threshold value and an amount of time the second trend is below the fifth threshold value.

6. The method of claim 4, wherein determining the risk of developing AKI comprises:
determining, by the processing circuitry and based on the second signal, a third trend in the output of the fluid over time; and
comparing, by the processing circuitry, the third trend to a sixth threshold value,
wherein determining the risk of developing AKI is based on the comparison.

7. The method of claim 6, wherein determining the risk of developing AKI is further based on a third amount of time the third trend is below the sixth threshold value.

8. The method of claim 3, wherein the first threshold value is based on the first baseline value and the second threshold value is based on the second baseline value.

9. The method of claim 3, wherein determining the first baseline value comprises:
determining, by the processing circuitry and based on the first signal, a first measure of dissolved oxygen in the fluid that is below a predetermined threshold value; and
averaging, by the processing circuitry, the first measure and measures of the dissolved oxygen in the fluid prior to the first measure.

10. The method of claim 1, wherein determining the first baseline value comprises:
averaging, by the processing circuitry, measures of the dissolved oxygen during a time period immediately before cardiopulmonary bypass surgery occurring to a patient.

11. The method of claim 1, wherein determining the first baseline value comprises:
determining, by the processing circuitry, at least two measures of the amount of dissolved oxygen in the fluid based on the first signal; and
applying, by the processing circuitry, at least one of an exponential decay or a non-linear regression to the at least two measures of the amount of dissolved oxygen in the fluid.

12. The method of claim 1, wherein determining the second baseline value comprises:
determining, by the processing circuitry, at least two measures of the amount of dissolved oxygen in the fluid based on the first signal;
determining, by the processing circuitry, at least two measures of the output of the fluid based on the second signal; and
applying, by the processing circuitry, at least one of an exponential decay or a non-linear regression to at least one of: a) the at least two measures of the amount of dissolved oxygen in the fluid based on the first signal; b) the at least two measures of the output of the fluid based on the second signal; or c) at least two measures of the total oxygen output in the fluid based on the at least two measures of the dissolved oxygen in the fluid and the at least two measures of the amount of dissolved oxygen in the fluid.

13. The method of claim 1, wherein the fluid is urine and the urine is output from a bladder of a patient.

14. A device comprising:
memory; and
processing circuitry communicatively coupled to the memory, the processing circuitry being configured to:
determine a first baseline value of dissolved oxygen in a fluid;
determine a second baseline value of a total oxygen output in the fluid;
receive, from a first sensor, a first signal indicative of an amount of dissolved oxygen in the fluid;
receive, from a second sensor, a second signal indicative of the output of the fluid; and
determine a risk of developing acute kidney injury (AKI) based at least in part on the first baseline value, the second baseline value, the first signal, and the second signal.

15. The device of claim 14, wherein the processing circuitry is further configured to:
determine a measure of total oxygen output in the fluid based on the first signal and the second signal,
wherein determining the risk of developing AKI further comprises determining the risk of developing AKI based on the measure of total oxygen output.

16. The device of claim 15, wherein as part of determining the risk of developing AKI, the processing circuitry is configured to:
determine the amount of dissolved oxygen in the fluid based on the first signal;
compare the amount of dissolved oxygen in the fluid to a first threshold value;
determine the output of the fluid based on the second signal;
compare the output of the fluid to a second threshold value; and
compare the measure of total oxygen output to a third threshold value,
wherein the risk of developing AKI is based on the comparisons.

17. The device of claim 16, wherein as part of determining the risk of developing AKI, the processing circuitry is configured to:
determine, based on the first signal, a first trend in the amount of dissolved oxygen in the fluid over time;
determine, based on the first signal and the second signal, a second trend in the measure of total oxygen output in the fluid;
compare the first trend to a fourth threshold value; and
compare the second trend to a fifth threshold value,
wherein determining the risk of developing AKI is based on the comparisons.

18. The device of claim 17, wherein the processing circuitry is configured to determine the risk of developing AKI further based on a first amount of time the first trend is below the fourth threshold value and an amount of time the second trend is below the fifth threshold value.

19. The device of claim 17, wherein as part of determining the risk of developing AKI, the processing circuitry is configured to:
determine, based on the second signal, a third trend in the output of the fluid over time; and
compare the third trend to a sixth threshold value,
wherein determining the risk of developing AKI is based on the comparison.

20. The device of claim 19, wherein determining the risk of developing AKI is further based on a third amount of time the third trend is below the sixth threshold value.

21. The device of claim 16, wherein the first threshold value is based on the first baseline value and the second threshold value is based on the second baseline value.

22. The device of claim 16 wherein as part of determining the first baseline value, the processing circuitry is configured to:
determine, based on the first signal, a first measure of dissolved oxygen in the fluid that is below a predetermined threshold value; and
average the first measure and measures of the dissolved oxygen in the fluid prior to the first measure.

23. The device of claim 14, wherein as part of determining the first baseline value, the processing circuitry is configured to:
average measures of the dissolved oxygen during a time period immediately before cardiopulmonary bypass surgery occurring to a patient.

24. The device of claim 14, wherein as part of determining the first baseline value, the processing circuitry is configured to:
determine at least two measures of the amount of dissolved oxygen in the fluid based on the first signal; and
apply at least one of an exponential decay or a non-linear regression to the at least two measures of the amount of dissolved oxygen in the fluid.

25. The device of claim 14, wherein as part of determining the second baseline value, the processing circuitry is configured to:
determine at least two measures of the amount of dissolved oxygen in the fluid based on the first signal;
determine at least two measures of the output of the fluid based on the second signal; and
apply at least one of an exponential decay or a non-linear regression to at least one of:
a) the at least two measures of the amount of dissolved oxygen in the fluid; b) the at least two measures of the output of the fluid based on the second signal; or c) at least two measures of the total oxygen output in the fluid based on the at least two measures of the dissolved oxygen in the fluid and the at least two measures of the amount of dissolved oxygen in the fluid.

26. The device of claim 14, wherein the fluid is urine and the urine is output from a bladder of a patient.

27. A device comprising:
memory; and
processing circuitry communicatively coupled to the memory, the processing circuitry being configured to:
determine at least two measures of an amount of dissolved oxygen in a fluid based on a first signal;
apply, to determine a first baseline value of dissolved oxygen in the fluid, at least one of an exponential decay or a non-linear regression to the at least two measures of the amount of dissolved oxygen in the fluid;
determine at least two measures of the output of the fluid based on a second signal;
apply, to determine a second baseline value of a total oxygen output in the fluid, at least one of an exponential decay or a non-linear regression to at least one of: a) the at least two measures of the amount of dissolved oxygen in the fluid; b) the at least two measures of the output of the fluid based on the second signal; or c) at least two measures of the total oxygen output in the fluid based on the at least two measures of the dissolved oxygen in the fluid and the at least two measures of the amount of dissolved oxygen in the fluid; and
determine a risk of developing acute kidney injury (AKI) based at least in part on the first baseline value, the second baseline value, the first signal, and the second signal.
